(19) United States
(12) Patent Application Publication
(10) Pub. No.: US 2004/0235883 A1
(43) Pub. Date: Nov. 25, 2004

(54) Alkyl-substituted imidazopyridines for the treatment of gastrointestinal disorders

(76) Inventors: Jorg Senn-Bilfinger, Konstanz (DE); Peter Jan Zimmermann, Radolfzell (DE)

Correspondence Address: NATH & ASSOCIATES PLLC, 1030 FIFTEENTH STREET, N.W., SIXTH FLOOR, WASHINGTON, DC, 20005, US

(21) Appl. No.: 10/485,514

(22) PCT Filed: Jul. 31, 2002
(86) PCT No.: PCT/EP02/08498

(30) Foreign Application Priority Data
Aug. 3, 2001 (EP) 01118674.9

(51) Int. Cl. A61K 31/4745; C07D 471/14

(52) U.S. Cl. 514/292; 546/82

(57) ABSTRACT

Compounds of the formula 1, in which the substituents and symbols are as defined in the description are suitable for pre-venting and treating gastrointestinal disorders.

### FIELD OF APPLICATION OF THE INVENTION

The invention relates to novel compounds which are used in the pharmaceutical industry as active compounds for preparing medicaments.

### PRIOR ART

U.S. Pat. No. 4,468,400 describes tricyclic imidazo[1,2-a]pyridines having different ring systems fused to the imidazopyridine skeleton, which compounds are said to be suitable for treating peptic ulcer disorders. The International Patent Applications WO95/27714, WO 98/42707, WO 98/54188, WO 00/17200, WO 00/26217, WO 00/63211, WO01/72754 and WO01/72757 disclose inter alia tricyclic imidazopyridine derivatives having a very specific substitution pattern, which compounds are likewise said to be suitable for treating gastrointestinal disorders.—Kaminski et al., J. Med. Chem. 1991, 34, 533-541 and 1997, 40, 427-436 describe the synthesis of imidazo[1,2-a]pyridines and their use as antiulcer agents.

### DESCRIPTION OF THE INVENTION

The invention provides compounds of the formula 1

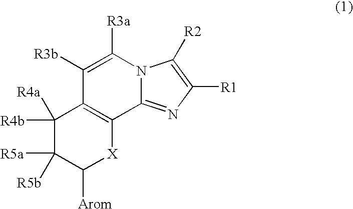

where

R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,

R2 is hydrogen, 1-4C-alkyl, 3-7C-cycloalkyl, 3-7C-cycloalkyl-1-4C-alkyl, 1-4C-alkoxycarbonyl, hydroxyl-4C-alkyl, halogen, 2-4C-alkenyl, 2-4C-alkynyl, fluoro-1-4C-alkyl or cyanomethyl,

R3a is hydrogen, halogen, fluoro-1-4C-alkyl, 1-4C-alkyl, 2-4C-alkenyl, 2-4C-alkynyl, 1-4C-alkoxy, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the radical —CO—NR31R32,

R3b is hydrogen, halogen, fluoro-1-4C-alkyl, 1-4C-alkyl, 2-4C-alkenyl, 2-4C-alkynyl, 1-4C-alkoxy, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the radical —CO—NR31R32,

where

R31 is hydrogen, 1-7C-alkyl, hydroxy-1-4C-alkyl or 1-4C-alkoxy-1-4C-alkyl and

R32 is hydrogen, 1-7C-alkyl, hydroxy-1-4C-alkyl or 1-4C-alkoxy-1-4C-alkyl,

or where

R31 and R32 together and including the nitrogen atom to which they are attached form a pyrrolidino, piperidino or morpholino radical,

one of the substituents R4a and R4b is hydrogen and the other is the radical R41,

where

R41 is 1-7C-alkyl, 2-7C-alkenyl, 3-7C-cycloalkyl, 1-4C-alkoxy-1-4C-alkyl, cyano-1-4C-alkyl, phenyl or phenyl-1-4C-alkyl,

one of the substituents R5a and R5b is hydrogen and the other is hydroxyl, 1-4C-alkoxy, oxo-substituted 1-4C-alkoxy, 3-7C-cycloalkoxy, 3-7C-cycloalkyl-1-4C-alkoxy, 1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, fully or predominantly halogen-substituted 1-4C-alkoxy or the radical R51,

where

R51 is a radical which, under physiological conditions, forms a hydroxyl group,

Arom is a R8-, R9-, R10- and R11-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,

where

R8 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,

R9 is hydrogen, 1-4C-alkyl, 1-4C-alkoxy, 1-4C-alkoxycarbonyl, halogen, trifluoromethyl or hydroxyl,

R10 is hydrogen, 1-4C-alkyl or halogen and

R11 is hydrogen, 1-4C-alkyl or halogen,

where

aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,

X is O (oxygen) or NH,

and their salts.

1-4C-Alkyl denotes straight-chain or branched alkyl radicals having 1 to 4 carbon atoms. Examples which may be mentioned are the butyl, Isobutyl, sec-butyl, tert-butyl, propyl, isopropyl, ethyl and methyl radicals.

3-7C-Cycloalkyl denotes cyclopropyl, cyclobutyl, cyclopentyl, cyclohexyl and cycloheptyl, among which cyclopropyl, cyclobutyl and cyclopentyl are preferred.

3-7C-Cycloalkyl-1-C-alkyl denotes one of the abovementioned 1-4C-alkyl radicals which is substituted by one of the abovementioned 3-7C-cycloalkyl radicals. Examples which may be mentioned are the cyclopropylmethyl, the cyclohexylmethyl and the cyclohexylethyl radicals.

1-4C-Alkoxy denotes radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

1-4C-Alkoxy-1-4C-alkyl denotes one of the abovementioned 1-4C-alkyl radicals which is substituted by one of the abovementioned 1-4C-alkoxy radicals. Examples which may be mentioned are the methoxymethyl, the methoxyethyl and the butoxyethyl radicals.

1-4C-Alkoxycarbonyl (—CO-1-4C-alkoxy) denotes a carbonyl group to which is attached one of the abovementioned 1-4C-alkoxy radicals. Examples which may be mentioned are the methoxycarbonyl (CH₃O—C(O)—) and the ethoxycarbonyl (CH₃CH₂O—C(O)—) radicals.

2-4C-Alkenyl denotes straight-chain or branched alkenyl radicals having 2 to 4 carbon atoms. Examples which may be mentioned are the 2-butenyl, 3-butenyl, 1-propenyl and the 2-propenyl (allyl) radicals.

2-4C-Alkynyl denotes straight-chain or branched alkynyl radicals having 2 to 4 carbon atoms. Examples which may be mentioned are the 2-butynyl, the 3-butynyl and, preferably, the 2-propynyl (propargyl radicals).

Fluoro-1-4C-alkyl denotes one of the abovementioned 1-4C-alkyl radicals which is substituted by one or more fluorine atoms. An example which may be mentioned is the trifluoromethyl radical.

Hydroxy-1-4C-alkyl denotes abovementioned 1-4C-alkyl radicals which are substituted by a hydroxyl group. Examples which may be mentioned are the hydroxymethyl, the 2-hydroxyethyl and the 3-hydroxypropyl radicals.

For the purpose of the invention, halogen is bromine, chlorine and fluorine.

1-4C-Alkoxy-1-4C-alkoxy denotes one of the abovementioned 1-4C-alkoxy radicals which is substituted by a further 1-4C-alkoxy radical. Examples which may be mentioned are the radicals 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl denotes one of the abovementioned 1-4C-alkoxy-1-4C-alkyl radicals which is substituted by one of the abovementioned 1-4C-alkoxy radicals. An example which may be mentioned is the radical 2-(methoxy)ethoxymethyl (CH₃—O—CH₂—CH₂—O—CH₂—).

Fluoro-1-4C-alkoxy-1-4C-alkyl denotes one of the abovementioned 1-4C-alkyl radicals which is substituted by a fluoro-1-4C-alkoxy radical. Here, fluoro-1-4C-alkoxy denotes one of the abovementioned 1-4C-alkoxy radicals which is fully or predominantly substituted by fluorine. Examples of fully or predominantly fluorine-substituted 1-4C-alkoxy which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals.

1-7C-Alkyl denotes straight-chain or branched alkyl radicals having 1 to 7 carbon atoms. Examples which may be mentioned are the heptyl, isoheptyl-(5-methylhexyl), hexyl, isohexyl-(4-methylpentyl), neohexyl-(3,3-dimethylbutyl), pentyl, isopentyl-(3-methylbutyl), neopentyl-(2,2-dimethylpropyl), butyl, isobutyl, sec-butyl, tert-butyl, propyl, isopropyl, ethyl and methyl radicals.

Oxo-substituted 1-4C-alkoxy denotes a 1-4C-alkoxy group which, instead of a methylene group, contains a carbonyl group. An example which may be mentioned is the 2-oxopropoxy group.

3-7C-Cycloalkoxy denotes cyclopropyloxy, cyclobutyloxy, cyclopentyloxy, cyclohexyloxy and cycloheptyloxy, among which cyclopropyloxy, cyclobutyloxy and cyclopentyloxy are preferred.

3-7C-Cycloalkyl-1-4C-alkoxy denotes one of the abovementioned 1-4C-alkoxy radicals which is substituted by one of the abovementioned 3-7C-cycloalkyl radicals. Examples which may be mentioned are the cyclopropylmethoxy, the cyclobutylmethoxy and the cyclohexylethoxy radicals.

3-7C-Cycloalkoxy-1-4C-alkoxy denotes one of the abovementioned 1-4C-alkoxy radicals which is substituted by one of the abovementioned 3-7C-cycloalkoxy radicals. Examples which may be mentioned are the cyclopropoxymethoxy, the cyclobutoxymethoxy and the cyclohexyloxyethoxy radicals.

3-7C-Cycloalkyl-1-4C-alkoxy-1-4C-alkoxy denotes one of the abovementioned 1-4C-alkoxy radicals which is substituted by one of the abovementioned 3-7C-cycloalkyl-1-4C-alkoxy radicals. Examples which may be mentioned are the cyclopropylmethoxyethoxy, the cyclobutylmethoxyethoxy and the cyclohexylethoxyethoxy radicals.

1-4C-Alkylcarbonyl denotes a radical which, in addition to the carbonyl group, contains one of the abovementioned 1-4C-alkyl radicals. An example which may be mentioned is the acetyl radical.

1-4C-Alkylcarbonyloxy denotes a 1-4C-alkylcarbonyl group which is attached to an oxygen atom. An example which may be mentioned is the acetoxy radical (CH₃CO—O—).

Fully or predominantely halogen-substituted 1-4C-alkoxy which may be mentioned are primarily chlorine- and/or, in particular, fluorine-substituted 1-4C-alkoxy radicals. Examples of halogen-substituted 1-4C-alkoxy which may be mentioned are the 2,2,2-trichloroethoxy, the hexachloroisopropoxy, the pentachloroisopropoxy, the 1,1,1-trichloro-3,3,3-trifluoro-2-propoxy, the 1,1,1-trichloro-2-methyl-2-propoxy, the 1,1,1-trichloro-2-propoxy, the 3-bromo-1,1,1-trifluoro-2-propoxy, the 3-bromo-1,1,1-trifluoro-2-butoxy, the 4-bromo-3,3,4,4-tetrafluoro-1-butoxy, the chlorodifluoromethoxy, the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals.

A radical R51 which forms a hydroxyl group under physiological conditions is to be understood as meaning a radical —OR′ from which, in the body of a human or animal, the group R′ is cleaved off hydrolytically forming the radical —OH and the non-toxic compound R′OH. Thus, the radical R′ can also be referred to as a hydroxyl protective group or as a prodrug radical. Such hydroxyl protective groups or prodrug radicals are known, inter alia, from the patent applications and patents DE 4308095, WO 95/14016, EP 694547, WO 95/11884, WO 94/05282 and U.S. Pat. No. 5,432,183. Radicals R′ having the general structure —C(O)R, —C(O)NRaRb, —P(O)ORaORb or —S(O)₂OR, where R, Ra and Rb denote any organic radicals or, if appropriate, hydrogen, may be mentioned by way of example.

In the context of the invention, exemplary radicals R′ which are to be particularly mentioned are the groups

—C(O)—NR12R13,

—C(O)-Alk-NR12R13,

—C(O)-Alk-C(O)—NR12R13,

—P(O)(OH)₂,

—S(O)₂NR12R13,

—C(O)—R12,

—C(O)—C₆H₃R14R15,

—C(O)—OR12,

—C(O)-Alk-C(O)—R12,

—C(O)-Alk-C(O)—OR12,

—C(O)—C(O)—R12,

—C(O)—C(O)—OR12 and

—CH₂—OR12,

where

Alk is 1-7C-alkylene,

R12 is hydrogen, 1-7C-alkyl or halogen-, carboxyl-, hydroxyl-, sulfo- (—SO₃H), sulfamoyl- (—SO₂NH₂), carbamoyl- (—CONH₂), 1-4C-alkoxy- or 1-4C-alkoxycarbonyl-substituted 1-4C-alkyl,

R13 is hydrogen or 1-4C-alkyl,

R14 is hydrogen, halogen, nitro, 1-4C-alkyl, 1-4C-alkoxy, 1-4C-alkoxycarbonyl, 1-C-alkoxycarbonylamino, 1-4C-alkoxy-1-4C-alkoxycarbonylamino or trifluoromethyl and

R15 is hydrogen, halogen, 1-4C-alkyl or 1-4C-alkoxy.

1-7C-Alkylene denotes straight-chain or branched 1-7C-alkylene radicals, for example the methylene (—CH₂—), ethylene (—CH₂CH₂—), trimethylene (—CH₂CH₂CH—), tetramethylene (—CH₂CH₂CH₂CH₂—), 1,2-dimethylethylene [—CH(CH₃)—CH(CH₃)—], 1,1-dimethylethylene [—C(CH₃)₂—CH₂—], 2,2-dimethylethylene [—CH₂—C(CH₃)₂—], isopropylidene [—C(CH₃)₂—], 1-methylethylene [—CH(CH₃)CH₂—], pentamethylene (—CH₂CH₂CH₂CH₂CH₂—), hexamethylene (—CH₂CH₂CH₂CH₂CH₂CH₂—) and heptamethylene (—CH₂CH₂CH₂CH₂CH₂CH₂CH₂—) radicals.

In this context, the groups —C(O)—N(CH₃)₂, —C(O)—N(C₂H₅)₂, —C(O)—NHC₂H₅, —C(O)—CH₂CH₂NH₂, —C(O)—(CH₂)₃NH₂, —C(O)—C(CH₃)₂NH₂, —C(O)—CH₂N(CH₃)₂, —C(O)—CH(NH₂)—CH(CH₃)₂, —C(O)—CH(NH₂)CH(CH₃)C₂H₅, —C(O)—(CH₂)₆C(O)N(CH₃)CH₂CH₂SO₃H, —P(O)(OH)₂, —(O)₂NH₂, —C(O)—H, —C(O)—C(CH₃)₃, —C(O)—CH₂CH₂COOH, —C(O)—CH₃, —C(O)—C₂H₅, —C(O)C₆H₅, —C(O)—C₆H₄-4—NO₂, —C(O)—C₆H₄-3-NO₂, —C(O)—C₆H₄—OCH₃, —C(O)C₆H₄-4—C(O)—OCH₃, —C(O)—OCH₃, —C(O)O-menthyl, —C(O)—CH₂—C(O)OCH₃, —C(O)—CH₂CH₂—C(O)OCH₃, —C(O)C(O)OCH₃, —C(O)—C(O)—OC₂H₅ and —CH₂OCH(CH₃)₂ are to be mentioned as exemplary radicals R′ to which particular emphasis is given.

1-4C-Alkoxycarbonylamino denotes an amino radical which is substituted by one of the abovementioned 1-4C-alkoxycarbonyl radicals. Examples which may be mentioned are the ethoxycarbonylamino and the methoxycarbonylamino radicals.

1-4C-Alkoxy-1-4C-alkoxycarbonyl denotes a carbonyl group to which one of the abovementioned 1-4C-alkoxy-1-4C-alkoxy radicals is attached. Examples which may be mentioned are the 2-(methoxy ethoxycarbonyl (CH₃—O—CH₂CH₂—O—CO—) and the 2-(ethoxy)ethoxycarbonyl (CH₃CH₂—O—CH₂CH₂—O—CO—) radicals.

1-4C-Alkoxy-1-4C-alkoxycarbonylamino denotes an amino radical which is substituted by one of the abovementioned 1-4C-alkoxy-1-4C-alkoxycarbonyl radicals. Examples which may be mentioned are the 2-(methoxy)ethoxycarbonylamino and the 2-(ethoxy)ethoxycarbonylamino radicals.

2-4C-Alkenyloxy denotes a radical which, in addition to the oxygen atom, contains a 2-4C-alkenyl radical. An example which may be mentioned is the allyloxy radical.

Aryl-1-4C-alkyl denotes an aryl-substituted 1-4C-alkyl radical. An example which may be mentioned is the benzyl radical.

Aryl-1-4C-alkoxy denotes an aryl-substituted 1-4C-alkoxy radical. An example which may be mentioned is the benzyloxy radical.

Mono- or di-1-4C-alkylamino radicals contain, in addition to the nitrogen atom, one or two of the abovementioned 1-4C-alkyl radicals. Preference is given to di-1-4C-alkylamino and in particular to dimethyl-, diethyl- or diisopropylamino.

1-4C-Alkylcarbonylamino denotes an amino group to which a 1-4C-alkylcarbonyl radical is attached. Examples which may be mentioned are the propionylamino (C₃H₇—C(O)NH—) and the acetylamino (acetamido, CH₃C(O)NH—) radicals.

Radicals Arom which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxymethoxyphenyl, 4-benzyloxy-3-methoxyphenyl, 3,5-bis-(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)₃-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-6-pyrimidine, 2-quinolinyl, 3-quinolinyl, 4-quinolinyl, 2-chloro 3-quinolinyl, 2-chloro-6-methoxy-3-quinolinyl, 8-hydroxy-2-quinolinyl and 4-isoquinolinyl.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired.

Pharmacologically unacceptable salts, which can be initially obtained, for example, as process products in the preparation of the compounds according to the invention on an industrial scale, are converted into the pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

The compounds of the formula 1 have centers of chirality in the skeleton in positions 7, 8 and 9. The Invention thus provides all feasible stereoisomers in any mixing ratio, including the pure enantiomers, which are the preferred subject matter of the invention.

Compounds which are to be emphasized are those compounds of the formula 1 where

R1 is hydrogen, 1-4C-alkyl, 3-7C-cycloalkyl, 1-4C-alkoxy-1-4C-alkyl, 2-4C-alkynyl or fluoro-1-4C-alkyl,

R2 is hydrogen, 1-4C-alkyl, halogen, 2-4C-alkenyl, 2-4C-alkynyl or fluoro-1-C-alkyl,

R3a is hydrogen,

R3b is hydrogen, halogen, 1-4C-alkyl or the radical —CO—NR31R32,

where

R31 is hydrogen, 1-7C-alkyl, hydroxy-1-4C-alkyl or 1-4C-alkoxy-1-4C-alkyl and

R32 is hydrogen, 1-7C-alkyl, hydroxy-1-4C-alkyl or 1-4C-alkoxy-1-4C-alkyl,

or where

R31 and R32 together and including the nitrogen atom to which they are attached are a pyrrolidino, piperidino or morpholino radical,

one of the substituents R4a and R4b is hydrogen and the other is the radical R41,

where

R41 is 1-4C-alkyl, 1-4C-alkoxy-1-4C-alkyl, cyano-1-4C-alkyl or phenyl-1-4C-alkyl,

one of the substituents R5a and R5b is hydrogen and the other is hydroxyl, 1-4C-alkoxy, oxo-substituted 1-4C-alkoxy, 3-7C-cycloalkoxy, 3-7C-cycloalkyl-1-4C-alkoxy or 1-4C-alkoxy-1-4C-alkoxy,

Arom is a R8-, R9-, R10- and R11-substituted mono- or bicyclic aromatic radical selected from the group consisting of phenyl, furanyl (furyl) and thiophenyl (thienyl),

where

R8 is hydrogen, 1-4C-alkyl, hydroxy-1-4C-alkyl, 1-4C-alkoxy, 1-4C-alkylcarbonyl, carboxyl, 1-4C-alkoxycarbonyl, halogen, hydroxyl, trifluoromethyl, 1-4C-alkylcarbonylamino, 1-4C-alkoxycarbonylamino, 1-4C-alkoxy-1-4C-alkoxycarbonylamino or sulfonyl,

R9 is hydrogen, 1-4C-alkyl, 1-C-alkoxy, 1-4C-alkoxycarbonyl, halogen, trifluoromethyl or hydroxyl,

R10 is hydrogen and

R11 is hydrogen,

X is O (oxygen) or NH,

and their salts.

Among the compounds according to the invention, emphasis is given to the optically pure compounds of the formula 1*

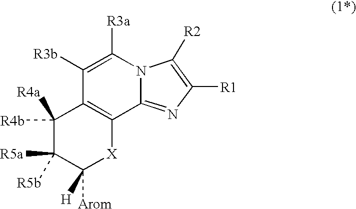

where the hydrogen atoms in positions 7 and 8 are preferably represented by the substituents R4a and R5b.

Among the compounds of the formula 1*, emphasis is given to those in which

R1 is hydrogen, methyl, cyclopropyl, methoxymethyl or trifluoromethyl,

R2 is hydrogen, methyl, chlorine, bromine, ethynyl or trifluoromethyl,

R3a is hydrogen,

R3b is hydrogen, fluorine, methyl or the radical —CO—N(CH₃)₂,

one of the substituents R4a and R4b is hydrogen and the other is the radical R41, where

R41 is 1-4C-alkyl, 1-C-alkoxy-1-4C-alkyl, cyano-1-4C-alkyl or phenyl-1-4C-alkyl,

one of the substituents R5a and R5b is hydrogen and the other is hydroxyl, methoxy, ethoxy, propoxy, isopropoxy, butoxy, methoxyethoxy, methoxypropoxy, methoxyethoxyethoxy, 2-oxopropoxy, cyclopropyloxy or cyclopropylmethoxy,

Arom is a phenyl radical,

X is O (oxygen) or NH,

and their salts.

Particular emphasis is given to compounds of the formula 1* in which

R1 is 1-4C-alkyl,

R2 is 1-4C-alkyl,

R3a is hydrogen,

R3b is hydrogen,

one of the substituents R4a and R4b is hydrogen and the other is the radical R41, where

R41 is 1-4C-alkyl, 1-4C-alkoxy-1-4C-alkyl, cyano-1-4C-alkyl, or phenyl-1-4C-alkyl,

one of the substituents R5a and R5b is hydrogen and the other is hydroxyl,

Arom is a phenyl radical,

X is O (oxygen) or NH,

and their salts.

The compounds of the formula 1 according to the invention can be prepared by reacting compounds M-R41, where R41 is as described above and M is the suitable radical of an organometallic compound, with compounds of the formula Z,

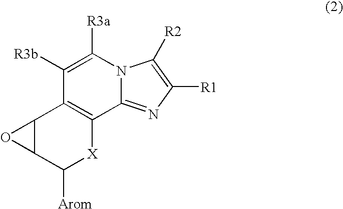

where R1, R2, R3a, R3b and Arom are as defined above and X is O (oxygen) or N-Prot, where Prot is an amino protective group which has to be cleaved off after the reaction. The reaction is carried out in a manner known per se to the person skilled in the art, depending on which radical R41 is to be introduced with the use of a customary organometallic compound M-R41 (such as, for example, methylithium, phenyllithium, 2,2-dimethylvinylmagnesium bromide etc.) in a suitable inert solvent, such as, for example acetonitrile or diethyl ether.

Suitable amino protective groups Prot are, in principle, all protective groups used for protecting amino acids in peptide and protein synthesis or for protecting other amines, for example in the synthesis of alkaloids and nucleotides (see, for example, T. W. Greene and P. G. M. Wuts, Protective groups in organic synthesis, 2^{nd} edition, 1991, John Wiley & Sons, Inc., pages 309-385). Exemplary protective groups which may be mentioned are the radicals 1-4C-alkylcarbonyl (for example acetyl), 1-4C-alkoxycarbonyl (for example butoxycarbonyl), benzyloxycarbonyl or nitrobenzenesulfenyl.

If the desired products are compounds of the formula 1 where X═NH, the amino protective group has to be cleaved off after the reaction of the organometallic compound M-R41 with the compound 2 where X═N-Prot. The amino protective group, for example the acetyl radical, can be cleaved off by heating the reaction product in ethanolamine in the presence of an auxiliary base, such as, for example, potassium carbonate.

The etherification of the hydroxyl group in the 8-position, which may follow, if desired, can be carried out, for example, as described in WO 00/17200. Any introduction of a prodrug radical R′ in the 8-position is carried out in an acylation reaction by reaction with compounds of the formula R′—Z in which Z is a suitable leaving group, for example a halogen atom. The reaction is carried out in a manner known per se, preferably in the presence of a suitable auxiliary base.

Compounds of the formula 1* which are to be emphasized are obtained by reacting an organometallic compound M-R41 with compounds of the formula 2*

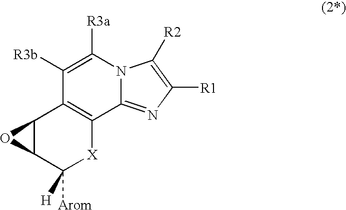

in which R1, R2, R3a, R3b and Arom are as defined above and X is O (oxygen) or N-Prot, where Prot is an amino protective group which has to be cleaved off after the reaction.

The compounds of the formula 2 in which R1, R2, R3a, R3b and Arom are as defined above and X is O (oxygen) or N-Prot can be prepared from the compounds of the formula 3

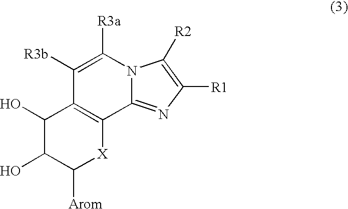

in which R1, R2, R3a, R3b and Arom are as defined above and X is O (oxygen) or N-Prot, as shown in Scheme 1 below in an exemplary manner for the compounds 2* and 3*:

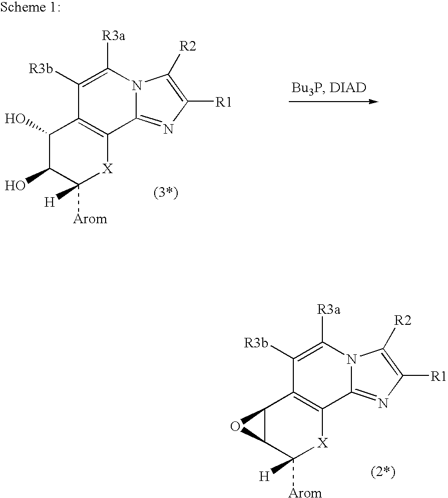

The conversion of the diol into the epoxide according to Scheme 1 is carried out in a manner known per se, for example using tributylphosphine and diisopropyl azodicarboxylate with cooling and under inert conditions (see, for example, J. Voss et al., Synthesis 2001, 229-234 or R. Mengel et al., Angew. Chem. 1978, 90, 725).

Compounds of the formula 3 are known, or they can be prepared as described in an exemplary maner in the examples below, or starting from corresponding starting materials and using analogous process steps (see, for example, WO 98/42707, WO 98/54188, WO 00/17200, WO 00/26217 and WO 00163211), or as shown quite generally in the schemes below.

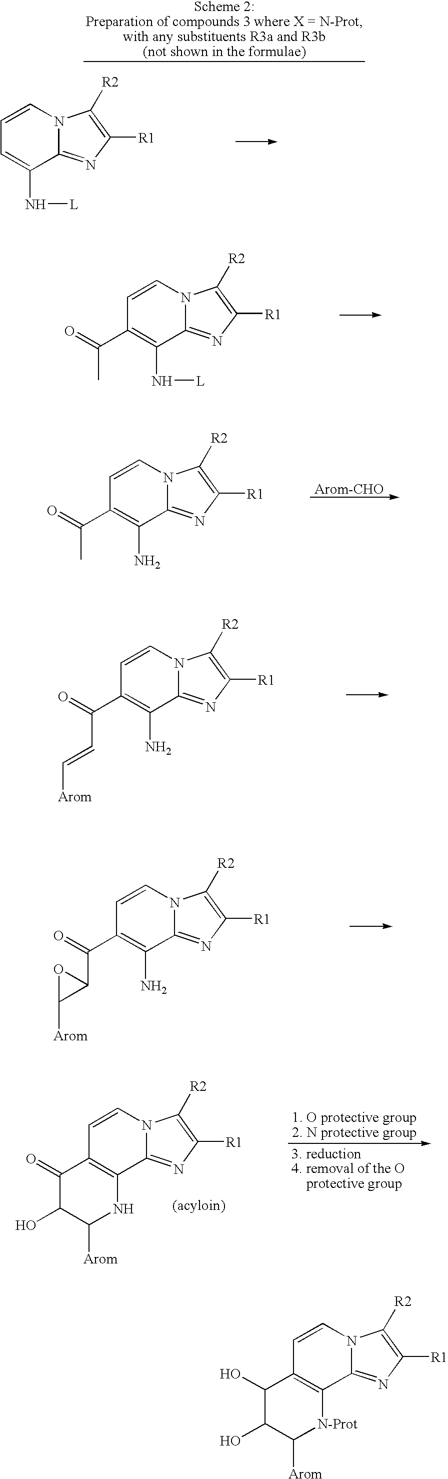

In the above scheme, L denotes any leaving group, for example a pivaloyl group. Introduction of the acetyl group and condensation with the aldehyde Arom-CHO are carried out in a manner known per se. The epoxidation is likewise carried out in a manner known per se, for example using hydrogen peroxide as epoxidizing agent. The introduction of O and N protective groups, the subsequent reduction and the removal of the O protective group that follows are likewise carried out in a manner known per se, for example as described in more detail in the examples below.

The preparation of compounds of the formula 3, shown by way of example for compounds of the formula 3* where X=O, with any substituents R3a and R3b, is advantageously carried out according to reaction Scheme 3 below.

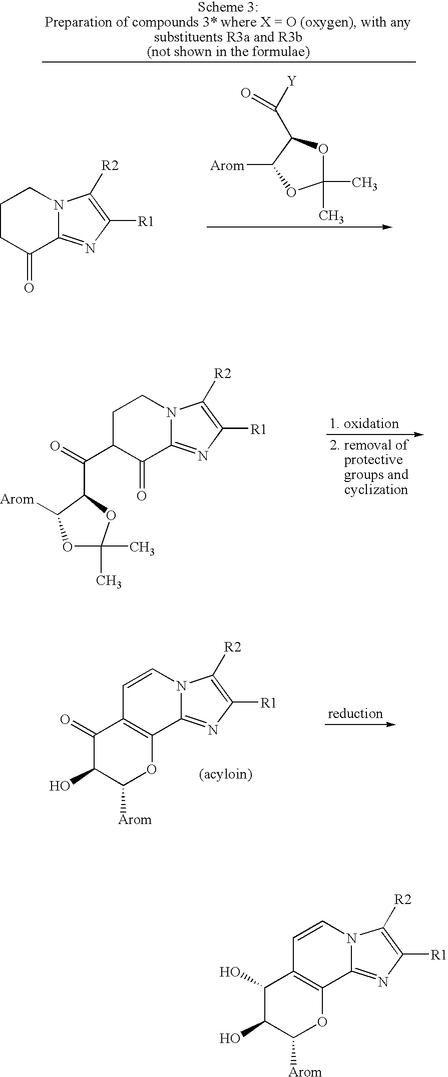

In Scheme 3 above, the enantioselective synthesis of a 7,8-diol of the formula 3* where X═O (oxygen) is shown in an exemplary manner. In Scheme 3, group Y is a suitable leaving group, for example a halogen atom, preferably chlorine, or a 1-4C-alkoxy group, preferably methoxy. The acylation is carried out in a manner familiar to the person skilled in the art, preferably using sodium bis(trimethylsilyl)amide or potassium bis(trimethylsilyl)amide, if the leaving group is a chlorine atom.

The oxidation that follows after the acylation is likewise carried out under customary conditions using the oxidizing agent chloranil, atmospheric oxygen, 2,3-dichloro-5,6-dicyano-p-benzoquinone or manganese dioxide. For the subsequent removal of protective groups and cyclization, certain conditions have to be met with respect to the auxiliary acid used. The auxiliary acid used is preferably formic acid.

The reduction to the diol is likewise—as in the reduction according to Scheme 2—carried out under standard conditions (see, for example WO 98/54188), where the reducing agent used is, for example, sodium borohydride, the use of which allows the given 7,8-trans-diol to be obtained in a diastereomeric purity of more than 90%. With respect to the targeted preparation and isolation of the pure enantiomers, reference is made, for example, to the relevant sections in WO 00117200.

The starting materials shown in Schemes 2 and 3 are known (see, for example, EP-A-299470, Kaminski et al., J. Med. Chem. 1985, 28, 876-892, 1989, 32, 1686-1700 and 1991, 34, 533-541 and Angew. Chem. 1996, 108, 589-591), or they can be prepared analogously to the known compounds, for example according to reaction Scheme 4 below.

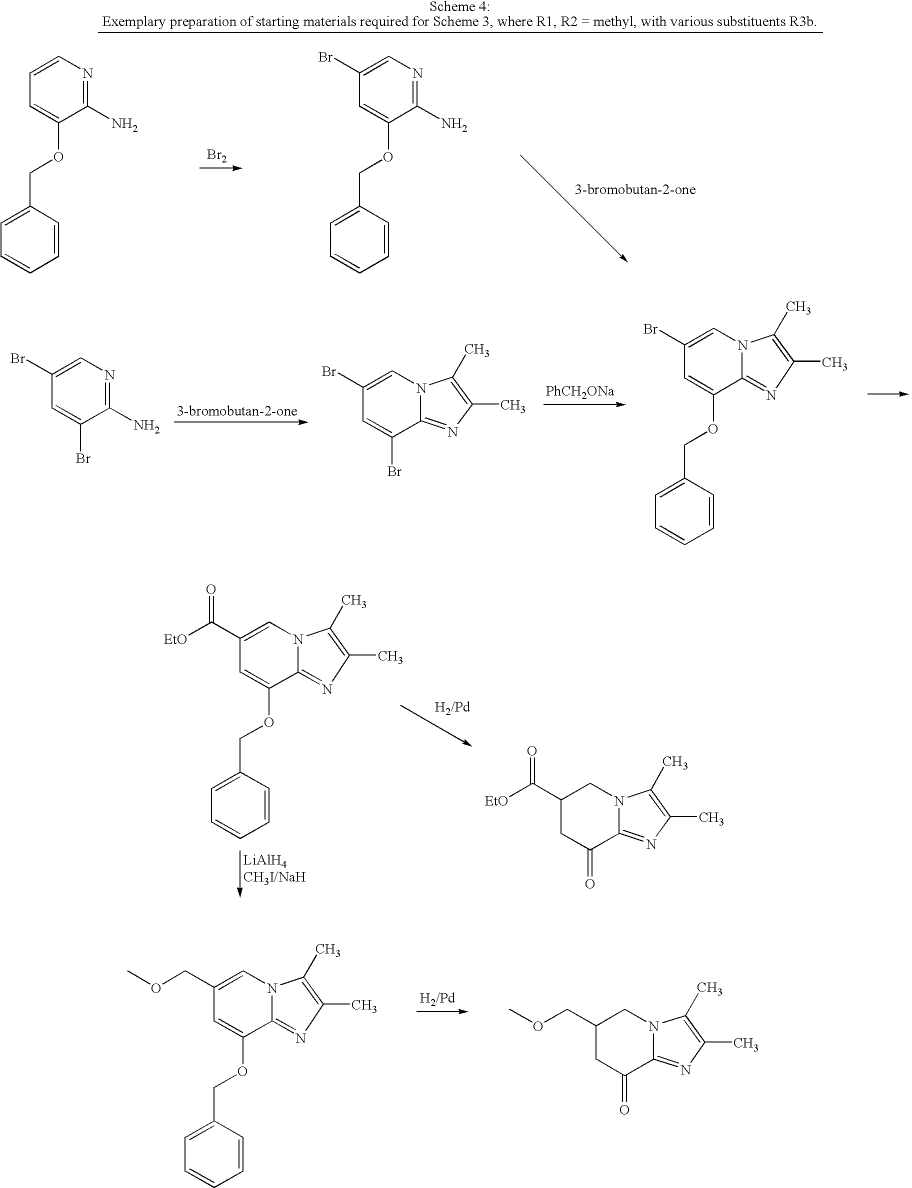

The conversion into the 8-benzyloxy-6-bromoimidazopyridines is carried-out in a manner familiar to the person skilled in the art. Conversion of the bromine atom into an ethyl ester radical can be effected by various routes, for example using the Heck reaction (with Pd(II), carbon monoxide and ethanol) or by metallation in the 6-position (with lithium or magnesium) and subsequent Grignard reaction. Metallation also offers the option to introduce other desired groups R3b into position 6, for example, fluorine, chlorine or the carboxyl group. Starting from the ester group, it is possible to introduce further desired groups R3b into position 6, for example hydroxy-1-4C-alkyl radicals (in particular the hydroxymethyl radical), by reducing the ester radical with lithium aluminum hydride, or 1-4C-alkoxy-1-4C-alkyl radicals (in particular 1-4C-alkoxymethyl radicals) by subsequent etherification as illustrated in Scheme 4.

The debenzylation/reduction is likewise carried out in a manner known per se, using, for example, hydrogen/Pd(0). If compounds where R3b=—CO—NR31R32 are desired, it is possible to carry out a corresponding derivatization in a manner known per se (conversion of an ester into an amide) at the stage of the 8-benzyloxy-6-ethoxycarbonyl compound or after the debenzylation/reduction, or alternatively also at a later stage, for example at the acyloin stage (see Schemes 2 and 3).

Starting materials with various substituents R1 and R2 are known, or they can be prepared in a known manner, analogously to known compounds, for example based on Scheme 4. Alternatively, derivatizations can also be carried out at the stage of the compounds 3. Thus, using compounds where R2=H, it is possible to prepare, for example, compounds where R2=CH₂OH (by Vilsmaler reaction and subsequent reduction), where R2=Cl or Br (by chlorination or bromination), where R2=propynyl (from the corresponding bromine compound using the Sonogashira reaction) or where R2=alkoxycarbonyl (from the corresponding bromine compound by Heck carbonylation).

The examples below serve to illustrate the invention in more detail without limiting it. Further compounds of the formula 1 whose preparation is not described explicitly can likewise be prepared in an analogous manner or in a manner known per to the person skilled in the art, using customary process techniques. The compounds named expressly as examples, and the salts of these compounds, are preferred subject matter of the invention. The abbreviation min stands for minute(s), h stands for hour(s) and m.p. stands for melting point.

### EXAMPLES

1. (8R,9R)-2,3-Dimethyl-9-phenyl-8-pivaloyloxy-7,8,9,10-tetrahydroimidazo[1.2-h][1.7]naphthyridin-7-one

Under argon and with exclusion of moisture, 140 g of (8R,9R)-2,3-dimethyl-8-hydroxy-9-phenyl-7,8,9,10-tetrahydroimidazo[1.2-h][1.7]naphthyridin-7-one (WO 00/17200, Example B1) are suspended in 1100 ml of dichloromethane. 70 ml of triethylamine and 2.5 g of 4-dimethylaminopyridine are added, and a solution of 62 ml of pivaloyl chloride in 70 ml of dichloromethane is then added dropwise such that the temperature of the reaction mixture does not exceed 30° C. (cooling with a water bath). The mixture is stirred overnight and then poured into 1 l of ice-water and stirred for another 10 min in the cold. The organic phase is separated off and the aqueous phase is extracted with dichloromethane (2×200 ml). The combined organic phases are washed with water (3×300 ml) until neutral, dried over sodium sulfate and evaporated. This gives 220 g of a yellowish oil which is crystallized using 600 ml of tert-butyl methyl ether. The mixture is stirred in the cold for 2 h and then filtered off, and the filter residue is washed with 200 ml of tert-butyl methyl ether and dried in a vacuum drying cabinet until the weight remains constant. This gives 175 g (97%) of the title compound as a slightly yellowish solid of m.p. 185-187° C.

2. (8R,9R)-10-Acetyl-2,3-dimethyl-9-phenyl-8-pivaloyloxy-7,8,9,10-tetrahydroimidazo[1.2-h]-[1.7]naphthyridin-7-one

Under argon and with exclusion of moisture, 175 g of (8R,9R)-2,3-dimethyl-9-phenyl-8-pivaloyloxy-7,8,9,10-tetrahydroimidazo[1.2-h][1.7]naphthyridin-7-one are dissolved with mechanical stirring in 2200 ml of toluene. With ice-cooling, half of the acetyl chloride (130 ml in total) is added dropwise over 30 min. The ice-bath is removed, and half of the triethylamine (250 ml in total) is added dropwise at 10° C. over a period of 40 min (the temperature rises to up to 30° C.). After 15 min at this temperature, the second half of the stated reagents is added as described above. With stirring, the mixture is then poured into 1 l of ice-water. The organic phase is separated off and the aqueous phase is extracted with ethyl acetate (2×200 ml). The combined organic phases are washed with water (3×400 ml), dried over sodium sulfate and evaporated. The yellow-brown residue is crystallized using 300 ml of tert-butyl methyl ether. After 1 h of stirring in the cold, the mixture is filtered off and the filter residue is washed with 200 ml of tert-butyl methyl ether and dried in a vacuum drying cabinet until the weight remains constant. 186 g (95%) of the title compound are isolated as a yellowish solid of m.p. 168-170° C.

3. (7R,8R,9R)-10-Acetyl-7-hydroxy-2,3-dimethyl-9-phenyl-8-pivaloyloxy-7,8,9,10-tetrahydroimidazo[1.2-h][1.7]naphthyridine

With mechanical stirring 165 g of (8R,9R)-10-acetyl-2,3-dimethyl-9-phenyl-8-pivaloyloxy-7,8,9,10-tetrahydroimidazo[1.2-h][1.7]naphthyridin-7-one are suspended in 2.0 l of isopropanol. With ice-cooling, 47.8 g of sodium cyanoborohydride are then introduced. 20 drops of Methyl Orange are added, and methanolic hydrogen chloride solution is then slowly added dropwise until the color remains red (about 150 ml, 1 h, warming of the reaction mixture to 16° C.). After a further 20 min, the mixture is poured into 1.51 of ice-water and 1 l of dichloromethane and neutralized with ammonia solution (25%). The organic phase is separated off and the aqueous phase is extracted with 250 ml of dichloromethane. The combined organic phases are re-extracted with water (3×1.5 l), dried over sodium sulfate and evaporated using a rotary evaporator. Coevaporation with acetone (3×) and drying of the residue under high vacuum gives 160 g (90%) of the title compound as a colorless foam of m.p. 103-105° C. which is used without further purification for the next step.

4. (7R,8R,9R)-10-Acetyl-7,8-dihydroxy-2,3-dimethyl-9-phenyl-7,8,9,10-tetrahydroimidazo-[1.2-h][1.7]naphthyridine

With stirring, 160 g of (7R,8R,9R)-10-acetyl-7-hydroxy-2,3-dimethyl-9-phenyl-pivaloyloxy-7,8,9,10-tetrahydroimidazo[1.2-h][1.7]naphthyridine are dissolved in 0.7 l of methanol, and 40 g of potassium carbonate are added. After about 10 min, the product begins to precipitate from the reaction mixture. After 1 h of stirring at room temperature, the mixture is poured into a solution of 200 g of ammonium chloride and 1.8 l of ice-water. The mixture is stirred for another 1 h at ice-bath temperature and the precipitated solid is then filtered off with suction and washed with a little methanol (80 ml). After drying in a vacuum cabinet at 50° C., 92.0 g (73%) of the title compound are isolated as a colorless solid of m.p. 260-261° C. which is used without further purification for the next step. Alternatively, the title compound can also be prepared according to Examples 5 and 6.

5. (8R,9R)-8,10-Diacetyl-2,3-dimethyl-9-phenyl-7,8,9,10-tetrahydroimidazo[1.2-h][1.7]naphthyridin-7-one

Under nitrogen and with exclusion of moisture, 50 g of (8R,9R-2,3-dimethyl-8-hydroxy-9-phenyl-7,8,9,10-tetrahydroimidazo[1.2-h][1.7]naphthyridin-7-one (WO 00/17200, Example B1) are dissolved in 450 ml of dichloromethane. At room temperature, initially half of the acetyl chloride (46.6 ml in total) is added dropwise. With ice-cooling, half of the triethylamine (45 ml in total) is then added dropwise over a period of 30 min. After 1 h of stirring at room temperature, the second half of the stated reagents is added as described. The mixture is then hydrolyzed using saturated sodium bicarbonate solution and water. The organic phase is separated off and the aqueous phase is extracted with dichloromethane. The combined organic phases are dried over magnesium sulfate and evaporated. The yellow-brown residue is coevaporated twice with toluene. 64 g of the title compound are isolated as a brown oil which is used without further purification for the next step.

6. (7R,8R,9R)-10-Acetyl-7,8-dihydroxy-2,3-dimethyl-9-phenyl-7,8,9,10-tetrahydroimidazo[1.2-h][1.7]naphthyridine

64 g of (8R,9R)-8,10-diacetyl-2,3-dimethyl-9-phenyl-7,8,9,10-tetrahydroimidazo[1.2-h][1.7]naphthyridin-7-one (crude product) are dissolved in 250 ml of methanol. With ice-cooling, 12.3 g of sodium borohydride are introduced. After 1 h of stirring, 23 g of potassium carbonate are added to the reaction mixture, which is then stirred at room temperature for another 2 h. The mixture is then poured into ice-water and the precipitate is filtered off with suction. The precipitate is washed with acetone and ether, and 37 g of the title compound are isolated.

7. (7S,8R,9R)-10-Acetyl-7,8-epoxy-2,3-dimethyl-7,8,9,10-tetrahydroimidazo-[1.2-h][1.7]naphthyridine

Under nitrogen, with exclusion of moisture and with ice-cooling, 98.0 g of (7R,8R,9R)-10-acetyl-7,8-dihydroxy-2,3-dimethyl-9-phenyl-7,8,9,10-tetrahydroimidazo[1.2-h][1.7]naphthyridine are suspended in 720 ml of dichloromethane. 79 ml of tributyl phosphine are added, and 60 ml of diisopropyl azodicarboxylate are then added dropwise at an internal temperature of 5° C. over a period of 45 min. After the addition has ended, the orange solution is stirred with ice-cooling for another 20 min. The reaction mixture is poured into 1 l of ice-water, the organic phase is separated off and the aqueous phase is extracted with dichloromethane (2×100 ml). The combined organic phases are washed with water (3×500 ml) and dried over sodium sulfate. When the organic phase is concentrated on a rotary evaporator (bath temperature <40° C.), the crystallization of the product starts when the volume has been reduced to about 1/10 of the original volume. With stirring, 500 ml of tert-butyl methyl ether are then slowly added dropwise. After 1 h of stirring with ice-cooling, the precipitate is filtered off with suction and washed with 200 ml of tert-butyl methyl ether. The product is dried at 40° C. in a vacuum drying cabinet until the weight remains constant. 86.0 g (92%) of the title compound are Isolated as a colorless solid of m.p. 205-206° C.

8. (7S,8S,9R)-8-Hydroxy-2,3,7-trimethyl-7,8,9,10-tetrahydroimidazo[1.2-h][1.7]naphthyridine

Under nitrogen, 3.75 ml of methylmagnesium bromide solution (3N in diethyl ether) are initially charged in 10 ml of tetrahydrofuran at −78° C. A solution of 1.5 g of (7S,8R,9R)-10-acetyl-7,8-epoxy-2,3-dimethyl-7,8,9,10-tetrahydroimidazo[1.2-h][1.7]naphthyridine in 10 ml of dichloromethane is then added dropwise over a period of 10 min. After 2 h, the reaction mixture is allowed to warm to 10° C. and poured into 20 ml of water. After addition of 5 ml of saturated ammonium chloride solution, the mixture is extracted with dichloromethane and the organic phase is washed with water and evaporated. The residue is purified by silica gel chromatography (diethyl ether/triethylamine 4:1). Following crystallization from diethyl ether, 200 mg (15%) of the title compound are isolated as a colorless solid of m.p. >240° C.

9. (7S,8S,9R)-7-Cyanomethyl-8-hydroxy-2,3-dimethyl-7,8,9,10-tetrahydroimidazo[1.2h]-[1.7]naphthyridine

Under nitrogen, 92 ml of lithium diisopropyldiamide solution (2 M) are initially charged at −78° C., and 9.7 ml of acetonitrile are added over a period of 30 min. After 1 h, a solution of 6 g of (7S,8R,9R)-10-acetyl-7,8-epoxy-2,3-dimethyl-7,8,9,10-tetrahydroimidazo[1.2-h][1.7]naphthyridine in 30 ml of dichloromethane is added dropwise at −50° C. over a period of 20 min. After 1 h at this temperature, the reaction mixture is allowed to warm to −20° C. and poured into 200 ml of water. The pH is adjusted to 9 using saturated ammonium chloride solution, the mixture is extracted with ethyl acetate and the organic phase is washed with water. After evaporation, the residue is purified by silica gel chromatography (diethyl ether/triethylamine 9:1). Crystallization from acetonitrile gives 2.04 g (34%) of the title compound as a light-yellow solid of m.p. 214-216° C.

10. (7S,8S,9R)-8-Hydroxy-2,3-dimethyl-7-propyl-7,8,9,10-tetrahydroimidazo[1.2-h][1.7]naphthyridine

Starting with (7S,8R,9R)-10-Acetyl-7,8-epoxy-2,3-dimethyl-7,8,9,10-tetrahydroimidazo[1.2h][1.7]naphthyridine and using propylmagnesium bromide, the title compound of m.p. 173-174° C. is obtained analogously to example 8.

11. (7R,8S,9R)-8-Hydroxy-2,3-dimethyl-7-(3-methoxypropyl)-7,8,9,10-tetrahydroimidazo-[1.2-h][1.7]naphthyridine

Starting with (7S,8R,9R)-10-acetyl-7,8-epoxy-2,3-dimethyl-7,8,9,10-tetrahydroimidazo[1.2-h][1.7]-naphthyridine and using 3-methoxypropylmagnesium bromide, the title compound of m.p. 140-142° C. is obtained analogously to example 8.

Commercial Utility

The compounds of the formula 1 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics), chemicals (e.g. ethanol), gastric acid or stress situations.

In their excellent properties, the compounds according to the invention surprisingly prove to be clearly superior to the compounds known from the prior art in various models in which the antiulcerogenic and the antisecretory properties are determined. On account of these properties, the compounds of the formula 1 and their pharmacologically acceptable salts are outstandingly suitable for use in human and veterinary medicine, where they are used, in particular, for the treatment and/or prophylaxis of disorders of the stomach and/or intestine.

A further subject of the invention are therefore the compounds according to the invention for use in the treatment and/or prophylaxis of the abovementioned diseases.

The invention likewise includes the use of the compounds according to the invention for the production of medicaments which are employed for the treatment and/or prophylaxis of the above-mentioned diseases.

The invention furthermore includes the use of the compounds according to the invention for the treatment and/or prophylaxis of the abovementioned diseases.

A further subject of the invention are medicaments which comprise one or more compounds of the formula 1 and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics (for example, tetracaine or procaine), and, if appropriate, also enzymes, vitamins or amino acids.

To be emphasized in this connection is in particular the combination of the compounds according to the invention with pharmaceuticals which inhibit acid secretion, such as, for example, H2 blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, meziocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

Pharmacology

The excellent gastric protective action and the gastric acid secretion-inhibiting action of the compounds according to the invention can be demonstrated in investigations on animal experimental models. The compounds according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No. | i.d. | (%) |
| 8 | 1 | 93 |
| 9 | 0.3 | 52 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tube just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01 N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion.

The body temperature of the animals was kept at a constant 37.8-38° C. by infrared irradiation and heat pads (automatic, stepless control by means of a rectal temperature sensor).

We claim:

1. A compound of the formula 1 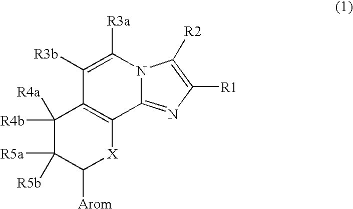
where
R1 is hydrogen, 1-4C-alkyl, 3-7C-cycloalkyl, 3-7-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxycarbonyl, hydroxy-1-4C-alkyl, halogen, 2-4C-alkenyl, 2-4C-alkynyl, fluoro-1-4C-alkyl or cyanomethyl,
R3a is hydrogen, halogen, fluoro-1-4C-alkyl, 1-4C-alkyl, 2-4C-alkenyl, 2-4C-alkynyl, 1-4C-alkoxy, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the radical —CO—NR31R32,
R3b is hydrogen, halogen, fluoro-1-4C-alkyl, 1-4C-alkyl, 2-4C-alkenyl, 2-4C-alkynyl, 1-4C-alkoxy, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the radical —CO—NR31R32,
where
R31 is hydrogen, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together and including the nitrogen atom to which they are attached form a pyrrolidino, piperidino or morpholino radical,
one of the substituents R4a and R4b is hydrogen and the other is the radical R41,
where
R41 is 1-7C-alkyl, 2-7C-alkenyl, 3-7C-cycloalkyl, 1-4C-alkoxy-1-4C-alkyl, cyano-1-4C-alkyl, phenyl or phenyl-1-4C-alkyl,
one of the substituents R5a and R5b is hydrogen and the other is hydroxyl, 1-4C-alkoxy, oxo-substituted 1-4C-alkoxy, 3-7C-cycloalkoxy, 3-7C-cycloalkyl-1-4C-alkoxy, 1-4C-alkoxy-1-4C-alkoxy, 3-7C-cycloalkoxy-1-4C-alkoxy, 3-7C-cycloalkyl-1-4C-alkoxy-1-4C-alkoxy, 1-4C-alkylcarbonyloxy, fully or predominantly halogen-substituted 1-4C-alkoxy or the radical R51,
where
R51 is a radical which, under physiological conditions, forms a hydroxyl group,
Arom is a R8-, R9-, R10- and R11-substituted mono- or bicyclic aromatic radical selected from the group consisting of phenyl, naphthyl, pyrrolyl, pyrazolyl, imidazolyl, 1,2,3-triazolyl, indolyl, benzimidazolyl, furanyl (furyl), benzofuranyl (benzofuryl), thiophenyl (thienyl), benzothiophenyl (benzothienyl), thiazolyl, isoxazolyl, pyridinyl, pyrimidinyl, quinolinyl and isoquinolinyl,
where
R8 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryloxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R9 is hydrogen, 1-4C-alkyl, 1-4C-alkoxy, 1-4C-alkoxycarbonyl, halogen, trifluoromethyl or hydroxyl,
R10 is hydrogen, 1-4C-alkyl or halogen and
R11 is hydrogen, 1-4C-alkyl or halogen,
where
aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
X is O (oxygen) or NH,
and its salts.

2. A compound of the formula 1 as claimed in claim 1, where
R1 is hydrogen, 1-4C-alkyl, 3-7C-cycloalkyl, 1-4C-alkoxy-1-4C-alkyl, 2-4C-alkynyl or fluoro-1-4C-alkyl,
R2 is hydrogen, 1-4C-alkyl, halogen, 2-4C-alkenyl, 2-4C-alkynyl or fluoro-1-4C-alkyl,
R3a is hydrogen,
R3b is hydrogen, halogen, 1-4C-alkyl or the radical —CO—NR31R32,
where
R31 is hydrogen, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together and including the nitrogen atom to which they are attached are a pyrrolidino, piperidino or morpholino radical,
one of the substituents R4a and R4b is hydrogen and the other is the radical R41,
where
R41 is 1-4C-alkyl, 1-4C-alkoxy-1-4C-alkyl, cyano-1-4C-alkyl or phenyl-1-4C-alkyl,
one of the substituents R5a and R5b is hydrogen and the other is hydroxyl, 1-4C-alkoxy, oxo-substituted 1-4C-alkoxy, 3-7C-cycloalkoxy, 3-7C-cycloalkyl-1-4C-alkoxy or 1-4C-alkoxy-1-4C-alkoxy,
Arom is a R8-, R9-, R10- and R11-substituted mono- or bicyclic aromatic radical selected from the group consisting of phenyl, furanyl (furyl) and thiophenyl (thienyl),
where
R8 is hydrogen, 1-4C-alkyl, hydroxy-1-4C-alkyl, 1-4C-alkoxy, 1-4C-alkylcarbonyl, carboxyl, 1-4C-alkoxycarbonyl, halogen, hydroxyl, trifluoromethyl, 1-4C-alkylcarbonylamino, 1-4C-alkoxycarbonylamino, 1-4C-alkoxy-1-4C-alkoxycarbonylamino or sulfonyl,
R9 is hydrogen, 1-4C-alkyl, 1-4C-alkoxy, 1-4C-alkoxycarbonyl, halogen, trifluoromethyl or hydroxyl,
R10 is hydrogen and
R11 is hydrogen,
X is O (oxygen) or NH,
and its salts.

3. A compound as claimed in claim 1, characterized by the formula 1* 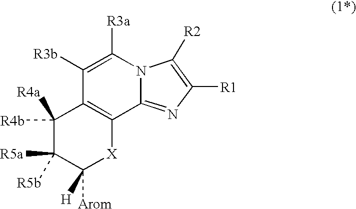
where
R1 is hydrogen, methyl, cyclopropyl, methoxymethyl or trifluoromethyl,
R2 is hydrogen, methyl, chlorine, bromine, ethynyl or trifluoromethyl,
R3a is hydrogen,
R3b is hydrogen, fluorine, methyl or the radical —CO—N(CH₃)₂,
one of the substituents R4a and R4b is hydrogen and the other is the radical R41, where
R41 is 1-4C-alkyl, 1-4C-alkoxy-1-4C-alkyl, cyano-1-4C-alkyl or phenyl-1-4C-alkyl
one of the substituents R5a and R5b is hydrogen and the other is hydroxyl, methoxy, ethoxy, propoxy, isopropoxy, butoxy, methoxyethoxy, methoxypropoxy, methoxyethoxyethoxy, 2-oxopropoxy, cyclopropyloxy or cyclopropylmethoxy,
Arom is a phenyl radical,
X is O (oxygen) or NH,
and its salts.

4. A compound as claimed in claim 1 of the formula 1* as claimed in claim 3, where
R1 is 1-4C-alkyl,
R2 is 1-4C-alkyl,
R3a is hydrogen,
R3b is hydrogen,
one of the substituents R4a and R4b is hydrogen and the other is the radical R41, where
R41 is 1-4C-alkyl, 1-4-alkoxy-1-4C-alkyl, cyano-1-4C-alkyl or phenyl-1-4C-alkyl,
one of the substituents R5a and R5b is hydrogen and the other is hydroxyl,
Arom is a phenyl radical,
X is O (oxygen) or NH,
and its salts.

5. A compound as claimed in claim 1 of the formula 1* as claimed in claim 3, where
R1 is 1-4C-alkyl,
R2 is 1-4C-alkyl,
R3a is hydrogen,
R3b is hydrogen,
one of the substituents R4a and R4b is hydrogen and the other is the radical R41, where
R41 is 1-4C-alkyl, 1-4C-alkoxy-1-4C-alkyl or cyano-1-4C-alkyl,
R5a is hydroxyl,
R5b is hydrogen,
Arom is a phenyl radical,
X is O (oxygen) or NH,
and its salts.

6. A medicament comprising a compound as claimed in claim 1 and/or a pharmacologically acceptable salt thereof together with customary pharmaceutical auxiliaries and/or excipients.

7. The use of compounds as claimed in claim 1 and their pharmacologically acceptable salts for the prevention and treatment of gastrointestinal disorders.
